(19) United States
(12) Patent Application Publication
(10) Pub. No.: US 2023/0323310 A1
(43) Pub. Date: Oct. 12, 2023

(54) METHOD OF PRODUCING THREE-DIMENSIONAL TISSUE BODY AND THREE-DIMENSIONAL TISSUE BODY

(71) Applicants: TOPPAN INC., Tokyo (JP); OSAKA UNIVERSITY, Osaka (JP)

(72) Inventors: Tomoko KUNITOMI, Taito-ku, Tokyo (JP); Shiro KITANO, Taito-ku, Tokyo (JP); Kei TSUKAMOTO, Taito-ku, Tokyo (JP); Michiya MATSUSAKI, Suita-shi, Osaka (JP)

(73) Assignees: TOPPAN INC., Tokyo (JP); OSAKA UNIVERSITY, Osaka (JP)

(21) Appl. No.: 18/043,507

(22) PCT Filed: Sep. 3, 2021
(86) PCT No.: PCT/JP2021/032525

§ 371 Date: Feb. 28, 2023

(30) Foreign Application Priority Data
Sep. 3, 2020 (JP) 2020-148332

(51) Int. Cl. C12N 5/071; C12N 5/077

(52) U.S. Cl. CPC C12N 5/069; C12N 5/0656; C12N 2513/00; C12N 2533/54; C12N 2501/91

(57) ABSTRACT

The present invention relates to a method for producing a three-dimensional tissue body, including: a step of obtaining a mixture by mixing a cationic substance and a fragmented extracellular matrix component with cells; and a step of culturing the cells after the step of obtaining the mixture.

### TECHNICAL FIELD

The present invention relates to a method for producing a three-dimensional tissue body and a three-dimensional tissue body.

### BACKGROUND ART

Three-dimensional tissue bodies have been shown to have applicability to various techniques such as regenerative medicine and drug assay systems. Various methods have been proposed so far as methods for producing a three-dimensional tissue body. For example, Patent Literature 1 discloses a method for producing a three-dimensional tissue body including: Step A of mixing cells with a cationic substance and an extracellular matrix component to obtain a mixture; Step B of collecting the cells from the obtained mixture to form a cell aggregate on a base material; and Step C of culturing the cells to obtain a three-dimensional tissue body.

### CITATION LIST

### Patent Literature

[Patent Literature 1] PCT International Publication No. WO2017/146124

### SUMMARY OF INVENTION

### Technical Problem

The thickness of a three-dimensional tissue body produced using a method in the related art may decrease when the culture period is lengthened.

An object of the present invention is to provide a method for producing a three-dimensional tissue body with suppressed reduction in thickness.

### Solution to Problem

The present invention relates to a method for producing a three-dimensional tissue body, comprising: a step of obtaining a mixture by mixing a cationic substance and a fragmented extracellular matrix component with cells; and a step of culturing the cells after the step of obtaining the mixture.

Since the production method according to the present invention comprises a step of obtaining a mixture by mixing a cationic substance and a fragmented extracellular matrix component with cells and a step of culturing the cells after the step of obtaining the mixture, a three-dimensional tissue body with suppressed reduction in thickness can be obtained.

In the production method according to the present invention, the cells may comprise at least interstitial cells and endothelial cells. Accordingly, the above-described effect is more significantly exhibited.

The fragmented extracellular matrix component may comprise fragmented collagen. The fragmented collagen may be defibrated collagen. In a case where the fragmented extracellular matrix component comprises fragmented collagen (for example, defibrated collagen), the above-described effect is more significantly exhibited.

In the production method according to the present invention, when obtaining the mixture, a polyelectrolyte is furthermore mixed with the cells in addition to the cationic substance and the fragmented extracellular matrix to incorporate the polyelectrolyte into the mixture. In this case, the above-described effect is more significantly exhibited.

The polyelectrolyte may comprise at least one selected from the group consisting of glycosaminoglycan, dextran sulfate, rhamnan sulfate, fucoidan, carrageenan, polystyrene sulfonic acid, polyacrylamide-2-methylpropanesulfonic acid, and polyacrylic acid. Accordingly, the above-described effect is more significantly exhibited.

A concentration of the polyelectrolyte in the mixture may be higher than 0 mg/mL and lower than or equal to 1.5 mg/mL.

In the production method according to the present invention, when obtaining the mixture, an extracellular matrix component may furthermore be mixed with the cells simultaneously or separately with respect to the cationic substance and/or the fragmented extracellular matrix.

The extracellular matrix component may comprise at least one selected from the group consisting of collagen, laminin, fibronectin, vitronectin, elastin, tenascin, entactin, fibrillin, and proteoglycan.

A mass ratio of the extracellular matrix component to the fragmented extracellular matrix component may be 2:1 to 1:50.

A total content of the extracellular matrix component and the fragmented extracellular matrix component in the mixture may be 0.005 mg/mL to 1.5 mg/mL.

The present invention also relates to a three-dimensional tissue body comprising: cells; a fragmented extracellular matrix component;

and a polyelectrolyte.

The polyelectrolyte may comprise at least one selected from the group consisting of glycosaminoglycan, dextran sulfate, rhamnan sulfate, fucoidan, carrageenan, polystyrene sulfonic acid, polyacrylamide-2-methylpropanesulfonic acid, and polyacrylic acid.

In the three-dimensional tissue body, a thickness retention rate represented by an equation below may be 80% or higher.

thickness retention rate=X₁/X₀×100 Equation:

[In the equation, X₁ represents a thickness of the three-dimensional tissue body after 4 days of culture, and X₀ represents a thickness of the three-dimensional tissue body at the start of culture.]

### Advantageous Effects of Invention

According to the present invention, it is possible to provide a method for producing a three-dimensional tissue body with suppressed reduction in thickness.

### BRIEF DESCRIPTION OF DRAWING

FIG. 1 is a photograph illustrating evaluation results of effects of reducing the thickness of three-dimensional tissue bodies.

### DESCRIPTION OF EMBODIMENTS

Hereinafter, embodiments of the present invention will be described in detail. However, the present invention is not limited to the following embodiments.

[Method for Producing Three-Dimensional Tissue Body]

The method for producing a three-dimensional tissue body according to the present embodiment comprises: a step of obtaining a mixture by mixing a cationic substance and a fragmented extracellular matrix component with cells (mixing step); and a step of culturing the cells after the step of obtaining the mixture (culture step).

In the present specification, the three-dimensional tissue body (cell structure) is a cell aggregate in which cells are three-dimensionally arranged through an extracellular matrix component and/or a fragmented extracellular matrix component and which is artificially produced through cell culture. The shape of the three-dimensional tissue body is not particularly limited, and examples thereof include a sheet shape, a spherical shape, an ellipsoidal shape, and a rectangular parallelepiped shape.

According to the method for producing a three-dimensional tissue body of the present embodiment, it is possible to produce a three-dimensional tissue body with suppressed reduction in thickness compared with methods in the related art. The mechanism by which such an effect is obtained is not particularly limited, but is thought that, for example, fragmented extracellular matrix components are aggregates of smaller extracellular matrix molecules, so they easily enter intercellular spaces and more easily strengthen intercellular adhesion.

<Mixing Step>

In the mixing step, a cationic substance and a fragmented extracellular matrix component are mixed with cells to obtain a mixture. In the mixing step, each of the above-described components can be mixed with cells simultaneously or separately. In the mixing step, mixing is performed so that at least parts of the cationic substance and the fragmented extracellular matrix component are incorporated into the mixture. In the mixing step, mixing with cells can be further performed so that at least a part of another component is incorporated into the mixture in addition to the cationic substance and the fragmented extracellular matrix component.

When obtaining the mixture, a polyelectrolyte may furthermore be mixed with the cells in addition to the cationic substance and the fragmented extracellular matrix to incorporate the polyelectrolyte into the mixture.

When obtaining the mixture, an extracellular matrix component may furthermore be mixed with the cells simultaneously or separately with respect to the cationic substance and/or the fragmented extracellular matrix.

The order of mixing in the above-described components used in the mixing step may be arbitrary. Any method can be employed as the method for mixing the above-described components with cells. The method for mixing the above-described components with cells may be, for example, a mixing method performed by adding cells to a liquid containing the above-described components.

The mixing of components used in the mixing step may be performed, for example, in a suitable container. A culture container for culturing cells can be used as the container. The container may be a container having a material and shape commonly used for culturing cells and microorganisms. Examples of materials of the container include, but are not limited to, glass, stainless steel, and plastic. Examples of the containers include, but are not limited to, dishes, tubes, flasks, bottles, and plates. As the container, for example, a container with a base material (permeable membrane) through which a liquid can pass but cells in the liquid do not pass can also be used. Examples of containers with a permeable membrane include, but are not limited to, cell culture inserts such as a Transwell (registered trademark) insert, a Netwell (registered trademark) insert, a Falcon (registered trademark) cell culture insert, and a Millicell (registered trademark) cell culture insert.

The components used in the mixing step may be respectively dissolved or dispersed in aqueous media and then mixed. Examples of aqueous media include water, physiological saline such as phosphate-buffered physiological saline (PBS) and liquid media such as a Dulbecco's Modified Eagle medium (DMEM).

(Cells)

Cells are not particularly limited, but cells may be derived from animals such as humans, monkeys, dogs, cats, rabbits, pigs, cattle, mice, and rats, for example. The origin of cells is also not particularly limited, but cells may be somatic cells derived from the bones, the muscles, the internal organs, the nerves, the brain, the bones, the skin, the blood, or the like, or may be reproductive cells. Furthermore, cells may be induced pluripotent stem cells (iPS cells) or embryonic stem cells (ES cells) or may be cultured cells such as primary culture cells, subcultured cells, and cell line cells.

Specific examples of cells include, but are not limited to, nerve cells, dendritic cells, immune cells, vascular endothelial cells (for example, human umbilical vein-derived vascular endothelial cells (HUVEC)), lymphatic endothelial cells, fibroblasts, cancer cells such as colorectal cancer cells (for example, human colorectal cancer cells (HT29)) and hepatoma cells, epithelial cells (for example, human gingival epithelial cells), keratinized cells, cardiomyocytes (for example, human iPS cell-derived cardiomyocytes (iPS-CM)), hepatocytes, islet cells, tissue stem cells, smooth muscle cells (for example, aortic smooth muscle cells (Aorta-SMC)). The cells may be used alone, or plural kinds of cells may be used in combination.

The cells may include interstitial cells. Interstitial cells are cells constituting supporting tissue of epithelial cells. Interstitial cells include fibroblasts, immune cells, pericytes, nerve cells, mast cells, epithelial cells, cardiomyocytes, hepatocytes, islet cells, tissue stem cells, and smooth muscle cells. In the case where the cells include interstitial cells, a three-dimensional tissue body with suppressed reduction in thickness is more easily obtained.

The cells preferably include endothelial cells. Endothelial cells are cells constituting the endothelium. Endothelial cells include the above-described vascular endothelial cells (for example, human umbilical vein-derived vascular endothelial cells (HUVEC)) and lymphatic endothelial cells, and sinusoidal endothelial cells. In the case where the cells include endothelial cells, a three-dimensional tissue body with suppressed reduction in thickness is more easily obtained.

The cells may include at least interstitial cells and endothelial cells from the viewpoint of more easily obtaining a three-dimensional tissue body with suppressed reduction in thickness. The ratio of the number of interstitial cells to the number of endothelial cells (number of interstitial cells:number of endothelial cells) may be, for example, 1:1.5 to 300:1.

The mixture comprises at least cells. The cell density in the mixture can be appropriately determined depending on the shape, the thickness, and the like of a target three-dimensional tissue body. For example, the cell density in the mixture may be 10³ to 10⁷ cells/mL, or may be 10⁴ to 10⁶ cells/mL.

(Cationic Substance)

A cationic substance is a substance having a cationic group. A cationic group is a cationic group (group having a positive charge) or a group that can be derivatized to a cationic group. Examples of cationic groups include an amino group (—NH₂), a substituted amino group (such as a monosubstituted amino group and a disubstituted amino group), and a quaternary ammonium group (quaternary ammonium cationic group).

Examples of cationic substances include, but are not limited to, trishydroxymethylaminomethane, (4-(2-hydroxyethyl)-1-piperazineethanesulfonic acid, ethanolamine, diethanolamine, triethanolamine, polyvinylamine, polyallylamine, polylysine, polyhistidine, and polyarginine.

In the mixing step, a cationic substance itself or a buffer solution (cationic buffer solution) containing a cationic substance can be incorporated to add the cationic substance to the above-described components used in the mixing step, for example. Examples of buffer solutions containing a cationic substance include a tris-hydrochloric acid buffer solution, a tris-maleic acid buffer solution, a bis-tris buffer solution, and a HEPES buffer solution. The concentration of a cationic substance in a cationic buffer solution may be, for example, 10 to 100 mM, 20 to 90 mM, 30 to 80 mM, 40 to 70 mM, or 45 to 60 mM, or may be 50 mM.

In a case of using a cationic buffer solution, the pH of the cationic buffer solution can be set to various pH values from the viewpoints of growth of cells, formation of cell aggregates, and the like. The pH of the cationic buffer solution may be 6.0 to 8.0 or 7.2 to 7.6. For example, the pH of the cationic buffer solution may be 7.0, 7.1, 7.2, 7.3, 7.4, 7.5, 7.6, 7.7, 7.8, 7.9, or 8.0. The pH of the cationic buffer solution is preferably 7.4.

(Extracellular Matrix Component and Fragmented Extracellular Matrix Component)

Extracellular matrix components are extracellular matrix molecule aggregates formed by a plurality of extracellular matrix molecules. Extracellular matrix molecules may be molecules that can be used to fill intercellular spaces, or may be a substance present outside cells in a multicellular organism. Arbitrary substances can be used as extracellular matrix molecules as long as these do not adversely affect growth of cells and formation of cell aggregates. Extracellular matrix molecules are preferably biocompatible. “Biocompatibility” means that excessive inflammation or the like is not caused when the extracellular matrix molecules are brought into contact with biological tissues. Examples of extracellular matrix molecules include, but are not limited to, collagen, laminin, fibronectin, vitronectin, elastin, tenascin, entactin, fibrillin, and proteoglycan. As extracellular matrix components, these extracellular matrix molecules may be used alone or in combination of two or more thereof.

Extracellular matrix components may, for example, comprise collagen or consist of collagen. In addition, in a case where extracellular matrix components comprise collagen, collagen functions as a scaffold for cell adhesion when it is used as a scaffold material during culturing cells, and formation of a three-dimensional cell structure is further promoted.

Extracellular matrix molecules may be modified extracellular matrix molecules or variants of extracellular matrix molecules or may be polypeptides such as chemically synthesized peptides. Extracellular matrix molecules may have a repetition of a sequence represented by Gly-X-Y which is characteristic to collagen. Here, Gly represents a glycine residue, and X and Y each independently represent an arbitrary amino acid residue. A plurality of Gly-X-Y's may be the same as or different from each other. If extracellular matrix molecules have a repetition of a sequence represented by Gly-X-Y, the degree of binding to a molecular chain arrangement is small. Therefore, for example, the function as a scaffold material during culturing cells is further improved. In the extracellular matrix molecules having the repetition of the sequence represented by Gly-X-Y, the proportion of the sequence represented by Gly-X-Y in the whole amino acid sequence may be 80% higher and preferably 95% or higher. In addition, the extracellular matrix molecules may be polypeptides having an RGD sequence. The RGD sequence is a sequence represented by Arg-Gly-Asp (arginine residue-glycine residue-aspartic acid residue). If the extracellular matrix molecules have an RGD sequence, cell adhesion is further promoted, and therefore the extracellular matrix molecules are even more suitable as scaffold materials during culturing cells, for example. Examples of extracellular matrix molecules having the sequence represented by Gly-X-Y and the RGD sequence include collagen, fibronectin, vitronectin, laminin, and cadherin.

Examples of collagen include fibrous collagen and non-fibrous collagen. Fibrous collagen means collagen that is a main component of collagen fibers, and specific examples thereof include type I collagen, type II collagen, and type III collagen. Examples of non-fibrous collagen include fibrous type IV collagen.

Examples of proteoglycans include, but are not limited to, chondroitin sulfate proteoglycans, heparan sulfate proteoglycans, keratan sulfate proteoglycans, and dermatan sulfate proteoglycans.

From the viewpoint of obtaining more significant effect of the present invention, an extracellular matrix component may comprise at least one selected from the group consisting of collagen, laminin, and fibronectin and preferably comprises collagen. Collagen is preferably fibrous collagen and more preferably type I collagen. Commercially available collagen may be used as fibrous collagen, and specific examples thereof include porcine skin-derived type I collagen manufactured by NH Foods Ltd.

An extracellular matrix component may be derived from animals. Examples of animal species from which extracellular matrix components are derived include, but are not limited to, humans, pigs, and cattle. A component derived from one type of animal may be used as an extracellular matrix component, or components derived from plural kinds of animals may be used in combination.

Fragmented extracellular matrix components can be obtained by fragmenting the above-described extracellular matrix components. The “fragmentation” means that an aggregate of extracellular matrix molecules is made smaller in size. The fragmentation may be performed under the conditions of cleaving the bond within extracellular matrix molecules or may be performed under the conditions of not cleaving the bond within extracellular matrix molecules. The fragmented extracellular matrix components may comprise defibrated extracellular matrix components in which the above-described extracellular matrix components are defibrated through application of physical force. Defibration is an aspect of fragmentation and is performed under the conditions of, for example, not cleaving the bond within extracellular matrix molecules.

The method for fragmenting extracellular matrix components is not particularly limited. As the method for defibrating extracellular matrix components, extracellular matrix components may be defibrated through application of physical force with an ultrasonic homogenizer, a stirring homogenizer, a high-pressure homogenizer, and the like, for example. In a case of using a stirring homogenizer, extracellular matrix components may be homogenized as they are or may be homogenized in an aqueous medium such as physiological saline. In addition, millimeter-sized and nanometer-sized defibrated extracellular matrix components can also be obtained by adjusting the time, the number of times of homogenizing or the like. Defibrated extracellular matrix components can also be obtained through defibration by repeating freezing and thawing.

Fragmented extracellular matrix components may include at least some defibrated extracellular matrix components. In addition, fragmented extracellular matrix components may consist of only defibrated extracellular matrix components. That is, fragmented extracellular matrix components may be defibrated extracellular matrix components. Defibrated extracellular matrix components preferably include defibrated collagen components. Defibrated collagen components preferably maintain a triple helix structure derived from collagen. Defibrated collagen components may be components partially maintaining a triple helix structure derived from collagen.

Examples of the shape of fragmented extracellular matrix components include a fibrous shape. The fibrous shape means a shape composed of a filamentous collagen component or a shape composed of a filamentous extracellular matrix component cross-linked between molecules. At least some fragmented extracellular matrix components may be fibrous. Fibrous extracellular matrix components include, for example, fine filamentous materials (fine fibers) formed by aggregating a plurality of filamentous extracellular matrix molecules, a filamentous material formed by further aggregating fine fibers, and one obtained by defibrating these filamentous materials. An RGD sequence is preserved without disruption in a fibrous extracellular matrix component.

The average length of fragmented extracellular matrix components may be 100 nm to 400 μm and 100 nm to 200 μm. In one embodiment, the average length of fragmented extracellular matrix components may be 5 μm to 400 μm, 10 μm to 400 μm, 22 μm to 400 μm, or 100 μm to 400 μm. In another embodiment, the average length of fragmented extracellular matrix components may be 100 μm or shorter, 50 μm or shorter, 30 μm or shorter, 15 μm or shorter, 10 μm or shorter, or 1 μm or shorter, and 100 nm or longer from the viewpoints of further improving redispersibility. Of all the fragmented extracellular matrix components, the average length of most of the fragmented extracellular matrix components is preferably within the above-described numerical ranges. Specifically, the average length of 95% of the fragmented extracellular matrix components of all the fragmented extracellular matrix components is preferably within the above-described numerical ranges. The fragmented extracellular matrix components are preferably fragmented collagen components having an average length within the above-described ranges and are more preferably defibrated collagen components having an average length within the above-described ranges.

The average diameter of the fragmented extracellular matrix components may be 10 nm to 30 μm, 30 nm to 30 μm, 50 nm to 30 μm, 100 nm to 30 μm, 1 μm to 30 μm, 2 μm to 30 μm, 3 μm to 30 μm, 4 μm to 30 μm, or 5 μm to 30 μm. The fragmented extracellular matrix components are preferably fragmented collagen components having an average diameter within the above-described ranges and are more preferably defibrated collagen components having an average diameter within the above-described ranges.

The average length and the average diameter of fragmented extracellular matrix components can be obtained by measuring each fragmented extracellular matrix component using an optical microscope and performing image analysis. In the present specification, the “average length” means an average value of the lengths of the measured samples in the longitudinal direction and the “average diameter” means an average value of the lengths of the measured samples in the direction orthogonal to the longitudinal direction.

At least some extracellular matrix components and/or fragmented extracellular matrix components (hereinafter also collectively referred to as “extracellular matrix components and the like”) may be cross-linked intermolecularly or intramolecularly. Extracellular matrix components and the like may be cross-linked within molecules constituting the extracellular matrix components and the like or may be cross-linked between molecules constituting the extracellular matrix components and the like.

The form of the cross-linking of extracellular matrix components and the like may be at least partly attributable to formation of hydrogen bonds between carboxyl groups and the like of the extracellular matrix molecules and ions of metal elements. The cross-linking of extracellular matrix components and the like may also include, for example, physical cross-linking by application of heat, ultraviolet rays, radiation, and the like and cross-linking by chemical cross-linking due to a cross-linking agent, an enzymatic reaction, and the like.

In the mixing step, for example, by incorporating fragmented extracellular matrix components themselves or a dispersion liquid composed of fragmented extracellular matrix components and an aqueous solvent in which the fragmented extracellular matrix components are dispersed, the fragmented extracellular matrix components can be mixed with the above-described components used in the mixing step. The aqueous medium used in the dispersion liquid may be a medium to be described below. Fragmented extracellular matrix components may be mixed with cells separately from a cationic substance. For example, fragmented extracellular matrix components may be mixed with cells after mixing a cationic substance with the cells.

The mixture contains at least fragmented extracellular matrix components. The content of the fragmented extracellular matrix components in the mixture can be appropriately determined depending on the shape, the thickness, and the like of a target three-dimensional tissue body. The content of the fragmented extracellular matrix components in the mixture may be, based on a total amount of mixture, for example, 0.005 mg/mL or more, 0.01 mg/mL or more, 0.025 mg/mL or more, 0.05 mg/mL or more, 0.10 mg/mL or more, 0.15 mg/mL or more, 0.20 mg/mL, 0.25 mg/mL, 0.30 mg/mL or more, 0.35 mg/mL or more, 0.40 mg/mL or more, or 0.45 mg/mL or more, and may be 1.5 mg/mL or less, 1.25 mg/mL or less, 1.0 mg/mL or less, 0.8 mg/mL or less, or 0.6 mg/mL or less. Increasing the content of the fragmented extracellular matrix components in the mixture tends to facilitate formation of a thicker three-dimensional tissue body. Decreasing the content of the fragmented extracellular matrix components in the mixture tends to facilitate suppression of reduction in thickness of the three-dimensional tissue body. If the content of the fragmented extracellular matrix components in the mixture is within the above-described ranges, a three-dimensional tissue body with suppressed reduction in thickness while having a moderate thickness is more easily formed.

In the mixing step, for example, by incorporating extracellular matrix components themselves or a solution containing extracellular matrix components and an aqueous medium, the extracellular matrix components can be mixed with the above-described components used in the mixing step. Extracellular matrix components may be dissolved in an appropriate solvent and used. Examples of solvents include, but are not limited to, water, a buffer solution, and acetic acid. Extracellular matrix components are preferably dissolved in a buffer solution or acetic acid. Extracellular matrix components may be mixed with cells simultaneously with a cationic substance, for example.

The mixture may comprise extracellular matrix components. The content of the extracellular matrix components in the mixture can be appropriately determined depending on the shape, the thickness, and the like of a target three-dimensional tissue body. The content of the extracellular matrix components in the mixture may be, based on a total amount of mixture, for example, 0.005 mg/mL or more, 0.01 mg/mL or more, 0.02 mg/mL or more, 0.03 mg/mL or more, or 0.04 mg/mL or more, and may be 1.5 mg/mL or less, 1.0 mg/mL or less, 0.1 mg/mL or less, 0.08 mg/mL or less, or 0.06 mg/mL or less. Increasing the content of the extracellular matrix components in the mixture tends to facilitate formation of a thicker three-dimensional tissue body. Decreasing the content of the extracellular matrix components in the mixture tends to facilitate suppression of reduction in thickness of the three-dimensional tissue body. If the content of the extracellular matrix components in the mixture is within the above-described ranges, a three-dimensional tissue body with suppressed reduction in thickness while having a moderate thickness is more easily formed.

The mass ratio of the extracellular matrix components to the fragmented extracellular matrix components (mass of extracellular matrix components:mass of fragmented extracellular matrix components) may be, for example, 2:1 to 1:50, 1:1 to 1:50, or 1:10 to 1:50.

In the mixing step, the total content of the extracellular matrix components and the fragmented extracellular matrix components in the mixture may be, based on a total amount of mixture, 0.005 mg/mL to 1.5 mg/mL, 0.01 mg/mL to 1.3 mg/mL, or 0.05 mg/mL to 1.2 mg/mL. Increasing the total content of the extracellular matrix components and the fragmented extracellular matrix components in the mixture tends to facilitate formation of a thicker three-dimensional tissue body. Decreasing the total content of the extracellular matrix components and the fragmented extracellular matrix components in the mixture tends to further facilitate suppression of reduction in thickness of the three-dimensional tissue body. If the total content of the extracellular matrix components and the fragmented extracellular matrix components in the mixture is within the above-described ranges, a three-dimensional tissue body with suppressed reduction in thickness while having a moderate thickness is more easily formed.

(Polyelectrolyte)

Polyelectrolytes are polymer compounds having polymer chains and dissociable functional groups. Examples of dissociable functional groups include a sulfate group (—SO₃H) and a carboxy group (—COOH). Polyelectrolytes may be, for example, polysaccharides into which a dissociable functional group is introduced or polymers containing a monomer (for example, acrylic acid and styrenesulfonic acid) having a dissociable functional group as a monomer unit. Examples of polysaccharides into which a dissociable functional group is introduced include sulfated polysaccharides. Polymers containing a monomer having a dissociable functional group as a monomer unit may be polymers containing only a monomer having a dissociable functional group as a monomer unit, or may be polymers containing other monomers in addition to a monomer having a dissociable functional group as monomer units. Examples of polyelectrolytes include, but are not limited to, glycosaminoglycans such as heparin, chondroitin sulfate (for example, chondroitin 4-sulfate and chondroitin 6-sulfate), heparan sulfate, dermatan sulfate, keratan sulfate, hyaluronic acid; and dextran sulfate, rhamnan sulfate, fucoidan, carrageenan, polystyrene sulfonic acid, polyacrylamide-2-methylpropanesulfonic acid, polyacrylic acid, and derivatives thereof. The polyelectrolytes may consist of one described above or may contain a combination of two or more thereof.

Polyelectrolytes are preferably glycosaminoglycan, more preferably comprise at least one selected from the group consisting of heparin, dextran sulfate, chondroitin sulfate, and dermatan sulfate, and are still more preferably heparin. In a case where a three-dimensional tissue body comprises a polyelectrolyte, excessive aggregation of extracellular matrix components and/or fragmented extracellular matrices can be more effectively suppressed, and as a result, a desired three-dimensional tissue body is more likely to be obtained. In a case where a three-dimensional tissue body comprises heparin, the effect is even more significant. As polyelectrolytes, derivatives of the above exemplified polyelectrolytes may be used, for example.

A polyelectrolyte may be dissolved in an appropriate solvent and used. Examples of solvents include, but are not limited to, water and a buffer solution. A polyelectrolyte may be dissolved together with the above-described cationic substance and used. A polyelectrolyte may be, for example, mixed with cells simultaneously with a cationic substance and extracellular matrix component.

The mixture may comprise a polyelectrolyte. The concentration of the polyelectrolyte in the mixture is not particularly limited as long as it does not adversely affect growth of cells and formation of cell aggregates. The concentration of the polyelectrolyte in the mixture may be, for example, higher than 0 mg/mL and lower than or equal to 1.5 mg/mL. The concentration of the polyelectrolyte in the mixture may be 0.005 mg/mL or more, 0.01 mg/mL or more, 0.02 mg/mL or more, 0.03 mg/mL or more, or 0.04 mg/mL or more, and may be 1.5 mg/mL or less, 1.0 mg/mL or less, 0.1 mg/mL or less, 0.08 mg/mL or less, or 0.06 mg/mL or less. The concentration of the polyelectrolyte in the mixture may be, for example, 0.025 mg/mL, 0.05 mg/mL, 0.075 mg/mL, or 0.1 mg/mL.

The mass ratio of the polyelectrolyte to the extracellular matrix component (mass of polyelectrolyte:mass of extracellular matrix component) in the mixture may be 1:2 to 2:1, 1:1.5 to 1.5:1, or may be 1:1.

The mixing step may include removing a liquid portion after at least one of the above-described components is mixed with cells. Techniques well known to those skilled in the art can be used as means for removing the liquid portion. The liquid portion may be removed through, for example, centrifugation and filtration. The conditions for centrifugation are not particularly limited as long as these do not adversely affect growth of cells and formation of cell aggregates. For example, a microtube containing cells and at least one of the above-described components can be subjected to centrifugation at 400×g for 1 minute at room temperature and the liquid portion can be separated from the precipitate to remove the liquid portion. After forming a precipitate through natural precipitation, the liquid portion may be removed.

The mixing step can be performed, for example, through a method including: mixing a liquid containing a cationic substance, a polyelectrolyte, an extracellular matrix component, and an aqueous medium with cells to obtain a cell-containing composition; removing at least a part of the liquid portion from the mixed liquid; and mixing a liquid containing a fragmented extracellular matrix component and an aqueous medium with the cell-containing composition from which at least a part of the liquid portion is removed.

The step of obtaining a mixture may include supplying (seeding) the mixture to a culture container. The above-described containers are exemplified as culture containers.

A step of collecting components other than a liquid component from a mixture to form a cell aggregate may be provided between the step of obtaining a mixture and a step of culturing cells. Techniques well known to those skilled in the art can be used as specific means for collecting components other a liquid component from a mixture to form a cell aggregate. For example, components other than a liquid portion may be collected through centrifugation, magnetic separation, or filtration. The conditions for centrifugation may be conditions that do not adversely affect growth of cells. For example, a mixture can be seeded in a cell culture insert and used for centrifugation at 400×g for 1 minute at 10° C. to collect cells. Alternatively, cells may be collected through natural precipitation. The time for natural precipitation may be, for example, 1 hour to 24 hours, and may be 24 hours from the viewpoint of ease of formation of cell aggregates.

<Culture Step>

In the culture step, cells are cultured after the mixing step under conditions in which at least some cells remain viable to form a three-dimensional tissue body. The culture of cells is usually performed on a base material. The base material may be, for example, the above-described permeable membrane. For culture of cells, the above-described containers with a permeable membrane can be used, for example.

The conditions for culturing cells may be any conditions as long as cells grow, and suitable culture conditions in the culture step can be set according to the types of cells. For example, the culture temperature may be 20° C. to 40° C. or may be 30° C. to 37° C. The pH of a medium may be 6 to 8 or may be 7.2 to 7.4. The culture time may be 1 day to 14 days, 7 days to 14 days, 14 days to 30 days, 30 days to 60 days, or 60 days to 90 days.

The liquid medium is not particularly limited, and a suitable medium can be selected depending on the types of cells to be cultured. Examples of the medium include an Eagle's MEM medium, DMEM, a Modified Eagle medium (MEM), a Minimum Essential medium, RPMI, and a GlutaMax medium. The medium may be a medium to which serum is added, or may be a serum-free medium. Furthermore, the liquid medium may be a mixed medium obtained by mixing two or more kinds of media with each other.

[Three-Dimensional Tissue Body]

The three-dimensional tissue body according to the present invention comprises at least cells, a fragmented extracellular matrix component, and a polyelectrolyte. The three-dimensional tissue body may further comprise an extracellular matrix component. At least some cells may come into contact with the extracellular matrix component and/or the fragmented extracellular matrix component. An aspect of the contact may include adhesion.

The three-dimensional tissue body according to the present embodiment can be obtained, for example, through the above-described method for producing a three-dimensional tissue body. The three-dimensional tissue body according to the present embodiment can be obtained, for example, through a method including: mixing a fragmented extracellular matrix component and a polyelectrolyte with cells simultaneously or separately to obtain a mixture; and centrifuging the mixture to culture the above-described cells, in this order.

The three-dimensional tissue body according to the present embodiment may have a thickness retention rate represented by an equation below of 80% or higher.

thickness retention rate(%)=X₁/X₀×100 Equation:

In the equation, X₁ represents a thickness of the three-dimensional tissue body after 4 days of culture (72 hours after the start of culture of the three-dimensional tissue body), and X₀ represents a thickness of the three-dimensional tissue body at the start of culture.

The thickness of a three-dimensional tissue body means the distance between both ends in a direction perpendicular to the main surface in a case where the three-dimensional tissue body has a sheet shape or a rectangular parallelepiped shape. In a case where the main surface is uneven, the thickness means the distance therebetween at the thinnest portion of the above-described main surface. In addition, in a case where a three-dimensional tissue body has a spherical shape, the thickness thereof means the diameter thereof. In addition, in a case where a three-dimensional tissue body has an ellipsoidal shape, the thickness thereof means the minor axis thereof. In a case where a three-dimensional tissue body has a substantially spherical shape or a substantially ellipsoidal shape and its surface is uneven, the thickness thereof means the shortest distance between two points where a straight line passing through the gravity center of the three-dimensional tissue body and the above-described surface intersect.

The thickness of a three-dimensional tissue body can be measured using a slice obtained by cutting the three-dimensional tissue body along the thickness direction (direction perpendicular to the main surface). The distance between both ends in the direction perpendicular to the main surface may be measured in a slice image passing through the center of the three-dimensional tissue body.

The thickness of a three-dimensional tissue body may be measured, for example, using an inverted microscope.

The thickness X₀ of a three-dimensional tissue body at the start of culture is a thickness of the three-dimensional tissue body at a point in time of 24 hours after formation of cell aggregates by collecting a mixture obtained by simultaneously or separately mixing a fragmented extracellular matrix component and a polyelectrolyte with cells. Cell culture may be performed in a medium at 37° C. for 24 hours after the formation of the cell aggregates. The cell culture may be performed in a CO₂ incubator (5% CO₂). A medium in which cells are likely to survive can be selected as a medium according to the types of cells.

The thickness X₀ of a three-dimensional tissue body at the start of culture may be 80 μm or more, 100 μm or more, or 110 μm or more, and may be 200 μm or less, 150 μm or less, or 120 μm or less.

The thickness X₁ of a three-dimensional tissue body after 4 days of culture is a thickness of the three-dimensional tissue body at a point in time of 96 hours after formation of cell aggregates (72 hours after the start of culture of the three-dimensional tissue body) by collecting a mixture obtained by simultaneously or separately mixing a fragmented extracellular matrix component and a polyelectrolyte with cells. For 96 hours after the formation of the cell aggregates, the culture may be performed under the same culture conditions as 24 hours after the formation of the cell aggregates. For example, cell culture may be performed in a medium at 37° C. for 96 hours after the formation of the cell aggregates. The cell culture may be performed in a CO₂ incubator (5% CO₂). A medium in which cells are likely to survive can be selected as a medium according to the types of cells.

The thickness X₁ of a three-dimensional tissue body after 4 days of culture may be 60 μm or more, 70 μm or more, or 80 μm or more, and may be 180 μm or less, 130 μm or less, or 100 μm or less.

The thickness retention rate may be, for example, 75% or higher, 80% or higher, or 82% or higher, and 100% or lower, 90% or lower, or 85% or lower.

The three-dimensional tissue body according to the present embodiment is a structure closer to biological tissue, and can be suitably used as a substitute for a laboratory animal and an implant material.

### EXAMPLES

Hereinafter, the present invention will be described in more detail based on test examples. However, the present invention is not limited to the following test examples.

NHDF (human neonatal dermal fibroblasts, manufacturer: Lonza, model number: CC-2509) and RFP-HUVECs (RFP-expressing human umbilical vein endothelial cells, manufacturer: Angio-Proteomie, model number: cAP-0001RFP) were prepared.

NHDF was cultured using D-MEM (Wako, 043-30085) containing 10% (v/v) fetal bovine serum (FBS, manufactured by Gibco, 10437028) and 1% (% v/v) penicillin-streptomycin (P/S) (Wako, 168-23191). RFP-HUVECs was cultured using EGM-2MV basic medium (Lonza, CC-3202).

The number of cells added per NHDF tissue (1 well) was 9×10⁵ cells. The number of cells added per RFP-HUVECs tissue (1 well) was 1.35×10⁵ cells.

A 20 mM Tris-hydrochloric acid buffer solution (pH 7.4) was prepared as a solution containing trishydroxymethylaminomethane (Tris). Heparin (Sigma, H3149-100KU) was dissolved in the 20 mM Tris-hydrochloric acid buffer solution (pH 7.4) to obtain a solution containing heparin and Tris. The content of heparin was 0.1 mg/mL based on the total amount of solution.

Collagen (collagen type I, manufactured by Nippi Incorporated) was dissolved in 5 mM acetic acid solution to obtain a collagen solution. The content of collagen was 0.1 mg/mL based on the total amount of solution.

50 mg of collagen type I (manufactured by NH Foods Ltd., porcine skin-derived) was suspended in 5 mL of 1×PBS (pH 7) and homogenized for 6 minutes at room temperature using a stirring homogenizer to obtain a fragmented collagen (defibrated collagen) solution. The average diameter of fragmented collagen in the obtained solution was 5.03±3.11 μm (N=25).

The fragmented collagen was dispersed in a medium (DMEM medium containing 10% FBS and 1% antibiotics) to prepare a fragmented collagen dispersion liquid.

### Example 1

NHDF and HUVEC were mixed with an equal amount mixture of a collagen solution and a solution containing heparin and Tris so that the final concentration of each of collagen and heparin was 0.05 mg/mL to prepare a cell suspension containing NHDF, HUVEC, collagen, Tris, and heparin. The obtained cell suspension was centrifuged at 1000×g for 1 minute at room temperature, a supernatant was removed, and then a fragmented collagen dispersion liquid was mixed therewith to a final concentration of 0.5 mg/mL.

The obtained mixture was seeded into a Transwell insert (96-well dedicated container, manufactured by Corning Inc., product number: 7369, pore size of 0.4 μm, polyester membrane). After adding an appropriate amount of universal medium (DMEM medium containing 10% FBS and 1% antibiotics) to a Transwell insert, centrifugation was performed for 1 minute under the condition of 400×g. Thereafter, the container was placed in a CO₂ incubator (37° C., 5% CO₂) to culture the cells under the conditions of 37° C. and 5% CO₂. 24 Hours after centrifugation was regarded as a start time point of culture. 96 Hours after centrifugation was regarded as an end time point of culture. A three-dimensional tissue body used for measurement was one in which there is no peeling from the edge of the insert.

Hematoxylin-eosin staining (HE staining) was performed on the slice specimen prepared from the cultured tissue. The thickness of the HE-stained three-dimensional tissue body at the start of culture and at the end of culture was measured using an inverted microscope (manufactured by Olympus Corporation). The thinnest portion of the specimen in the thickness direction was measured as a thickness of the three-dimensional tissue body.

The thickness retention rate of the three-dimensional tissue body was evaluated based on a thickness retention rate calculated by the following equation. The results are shown in Table 1.

Thickness retention rate(%)=(thickness of three-dimensional tissue body after completion of culture (72 hours after start of culture)/thickness of three-dimensional tissue body at start of culture)×100 Equation:

In the evaluation sample prepared by the method of Example 1, the thickness of the tissue at the start of culture was 97 μm and the thickness of the tissue at the end of culture was 81 μm.

### Example 2

NHDF and HUVEC were mixed with a solution containing heparin and Tris so that the final concentration of heparin was 0.05 mg/mL to prepare a cell suspension containing NHDF, HUVEC, Tris, and heparin. The obtained cell suspension was centrifuged at 1000×g for 1 minute at room temperature, a supernatant was removed, and then a fragmented collagen dispersion liquid was mixed therewith to a final concentration of 0.5 mg/mL. Cell culture and evaluation were performed in the same method as in Example 1 using the obtained mixture.

The thickness of a three-dimensional tissue body prepared by the method of Example 2 at the start of culture was 115 μm and the thickness thereof at the end of culture was 78 μm.

### Comparative Example 1

NHDF and HUVEC were mixed with an equal amount mixture of a collagen solution and a heparin-containing Tris solution so that the final concentration of each of collagen and heparin was 0.05 mg/mL to prepare a cell suspension containing NHDF, HUVEC, collagen, Tris, and heparin. The obtained cell suspension was centrifuged at 1000×g for 1 minute at room temperature, a supernatant was removed, and then a medium (DMEM medium containing 10% FBS and 1% antibiotics) was mixed therewith. Cell culture and evaluation were performed in the same method as in Example 1 using the obtained mixture.

The thickness of a three-dimensional tissue body prepared by the method of Comparative Example 1 at the start of culture was 97 μm and the thickness thereof at the end of culture was 56 μm.

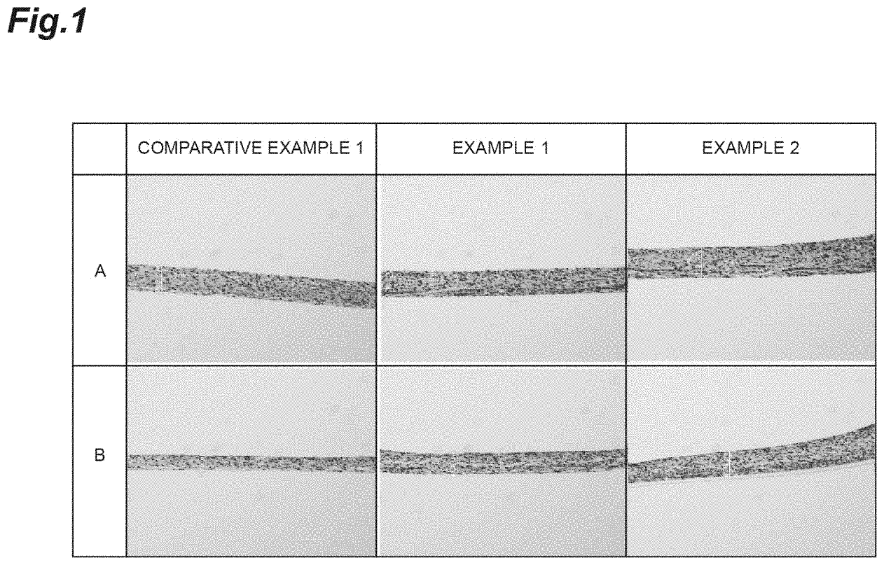

Table 1 shows the evaluation results of the thickness retention rate. The three-dimensional tissue body (Comparative Example 1) obtained by mixing 0.5 mg/mL of heparin and 0.5 mg/mL of collagen with cells was used as a reference body, and it was determined that the reduction in thickness is suppressed in a case where the thickness retention rate is larger than the reference body when the three-dimensional tissue bodies were cultured under the same conditions (medium, culture temperature, and culture period). In FIG. 1, A shows photographs illustrating the three-dimensional tissue bodies of Comparative Example 1 and Examples 1 and 2 at the start of culture. In FIG. 1, B shows photographs illustrating the three-dimensional tissue bodies of Comparative Example 1 and Examples 1 and 2 at the end of culture. The thickness of each linear blank portion in the photographs shown in FIG. 1 was measured.

TABLE 1

| | Comparative | Example | Example |
|---|---|---|---|
| | Example 1 | 1 | 2 |
| Collagen (mg/mL) | 0.05 | 0.05 | — |
| Fragmented collagen | — | 0.5 | 0.5 |
| (mg/mL) | | | |
| Thickness retention | 58 | 83 | 81 |
| rate (%) | | | |

In a case where fragmented collagen was used, the reduction in thickness was suppressed even after culture over a long period of time.

1. A method for producing a three-dimensional tissue body, comprising:
a step of obtaining a mixture by mixing a cationic substance and a fragmented extracellular matrix component with cells; and
a step of culturing the cells after the step of obtaining the mixture.

2. The method for producing a three-dimensional tissue body according to claim 1,
wherein the cells comprise at least interstitial cells and endothelial cells.

3. The method for producing a three-dimensional tissue body according to claim 1,
wherein the fragmented extracellular matrix component comprises fragmented collagen.

4. The method for producing a three-dimensional tissue body according to claim 3,
wherein the fragmented collagen is defibrated collagen.

5. The method for producing a three-dimensional tissue body according to claim 1,
wherein, when obtaining the mixture, a polyelectrolyte is furthermore mixed with the cells in addition to the cationic substance and the fragmented extracellular matrix to incorporate the polyelectrolyte into the mixture.

6. The method for producing a three-dimensional tissue body according to claim 5,
wherein the polyelectrolyte comprises at least one selected from the group consisting of glycosaminoglycan, dextran sulfate, rhamnan sulfate, fucoidan, carrageenan, polystyrene sulfonic acid, polyacrylamide-2-methylpropanesulfonic acid, and polyacrylic acid.

7. The method for producing a three-dimensional tissue body according to claim 5,
wherein a concentration of the polyelectrolyte in the mixture is higher than 0 mg/mL and lower than or equal to 1.5 mg/mL.

8. The method for producing a three-dimensional tissue body according to claim 1,
wherein, when obtaining the mixture, an extracellular matrix component is furthermore mixed with the cells simultaneously or separately with respect to the cationic substance and/or the fragmented extracellular matrix.

9. The method for producing a three-dimensional tissue body according to claim 8,
wherein the extracellular matrix component comprises at least one selected from the group consisting of collagen, laminin, fibronectin, vitronectin, elastin, tenascin, entactin, fibrillin, and proteoglycan.

10. The method for producing a three-dimensional tissue body according to claim 8,
wherein a mass ratio of the extracellular matrix component to the fragmented extracellular matrix component is 2:1 to 1:50.

11. The method for producing a three-dimensional tissue body according to claim 8,
wherein a total content of the extracellular matrix component and the fragmented extracellular matrix component in the mixture is 0.005 mg/mL to 1.5 mg/mL.

12. A three-dimensional tissue body comprising:
cells;
a fragmented extracellular matrix component; and
a polyelectrolyte.

13. The three-dimensional tissue body according to claim 12,
wherein the polyelectrolyte comprises at least one selected from the group consisting of glycosaminoglycan, dextran sulfate, rhamnan sulfate, fucoidan, carrageenan, polystyrene sulfonic acid, polyacrylamide-2-methylpropanesulfonic acid, and polyacrylic acid.

14. The three-dimensional tissue body according to claim 12,
wherein a thickness retention rate represented by an equation below is 80% or higher,
thickness retention rate=X₁/X₀×100 Equation:
[in the equation, X₁ represents a thickness of the three-dimensional tissue body after 4 days of culture, and X₀ represents a thickness of the three-dimensional tissue body at the start of culture].
